(19) United States
(12) Patent Application Publication
(10) Pub. No.: US 2023/0172591 A1
(43) Pub. Date: Jun. 8, 2023

(54) Liquid Sample Collection Devices, Systems, and Methods

(71) Applicant: Porex Corporation, Fairburn, GA (US)

(72) Inventors: Avi Robbins, Atlanta, GA (US); Gil Kan, Alpharetta, GA (US); Saman Mahdavi Shahidani, Lawrenceville, GA (US); Fernando Miquel Quispe Tardio, Lawrenceville, GA (US); Christopher John Ruf, Sandy Springs (US)

(21) Appl. No.: 18/076,716

(22) Filed: Dec. 7, 2022

Related U.S. Application Data
(60) Provisional application No. 63/265,079, filed on Dec. 7, 2021.

(51) Int. Cl. A61B 10/00

(52) U.S. Cl. CPC A61B 10/0051

(57) ABSTRACT

Liquid sample collection devices, systems, and methods. In one example, the system includes a liquid collection device, a cap for the liquid collection device, and a sealed reagent liquid container installed in the cap, in which depressing the cap relative to the liquid collection device unseals the reagent liquid container. In another example, the system includes a liquid collector and a collection chamber in which the collection chamber holds a reagent in a sealed condition and the liquid collector is actuable to disrupt a sealing membrane associated with the collection chamber. In another example, the system includes a liquid collector and a collection chamber in which the liquid collector holds a reagent in a sealed condition and in which the liquid collector is actuable to drain the reagent from the liquid collector into the collection chamber.

### CROSS REFERENCE TO RELATED APPLICATION

This application claims priority to U.S. provisional patent application Ser. No. 63/265,079 filed Dec. 7, 2022, the entire contents of which are hereby incorporated by this reference.

### RELATED FIELDS

According to certain embodiments of this disclosure, there is provided a liquid specimen collection device. Various embodiments implement oral fluid collection, extraction, storage, and/or testing. The device can be used to collect fluid, and to then extract and prepare the fluid for analysis, which can eliminate human error in collection and reduce complexity in diagnostic analysis. The disclosed device may be used to collect any liquid specimen. Any instance when liquid should be collected, stored, and tested may benefit from use of this disclosure.

### BACKGROUND

Oral fluid collection devices are widely used in the medical diagnostics industry for safe and convenient sample collection and disease identification. For examples, U.S. Pat. Nos. 9,198,641; 5,339,829; and 8,025,851 disclose devices that use a plunger, a collection pad, an adequacy indicator buffer, a compression seal, a filter, and a cartridge in various forms and combinations. There are also saliva collection systems manufactured by DNA Genotek, Ancestry, Oasis Diagnostics Corp., and Saliva Diagnostic Systems. These devices all have drawbacks and there remains significant room for improvement of this technology.

### SUMMARY

We describe in this patent several examples of liquid sample collection devices, systems, and methods configured to combine a collected liquid sample with a reagent, stabilizer, buffer, and/or additive (for brevity, all of which are referred to as a “reagent” in this patent) that has been retained in the device in a sealed condition prior to use. In some examples, the reagent is retained in the device in a sealed condition in the device's cap. In other examples, the reagent is retained in a collection chamber of the device in a sealed condition. In still other examples, the reagent is retained in the device's body in a sealed condition.

In one example, a liquid sample collection system may include a liquid collection device; a cap for the liquid collection device; a sealed reagent liquid container installed in the cap; in which depression of the cap relative to the liquid collective device unseals the reagent liquid container.

In some implementations of this example, the liquid collection device may include a collection pad and a collection chamber that is configured such that unsealing the reagent liquid container causes a reagent liquid to flow through the collection pad and into the collection chamber.

In some implementations of this example, the system may be configured such that depression of the cap compresses the collection pad against the sealed reagent liquid container to unseal the reagent liquid container.

In some implementations of this example, the system may be configured such that the collection pad collapses as it compresses against the sealed reagent liquid container.

In some implementations of this example, the sealed reagent liquid container may include a membrane, with the system configured such that compression of the collection pad against the sealed reagent liquid container dislodges the membrane.

In some implementations of this example, the cap may have a first open end for receiving the liquid collection device and a second open end for connecting to the sealed reagent liquid container.

In some implementations of this example, the system may be configured such that compression of the collection pad against the sealed reagent liquid container translates the sealed reagent liquid container vertically inside the cap.

In some implementations of this example, the sealed reagent liquid container may be held inside the cap by a friction fit between the sealed reagent liquid container and the cap.

In some implementations of this example, the cap may include an internal protrusion extending downward towards the sealed reagent liquid container, with the system configured such that compression of the collection pad against the sealed reagent liquid container translates the sealed reagent liquid container upward into contact with the internal protrusion to unseal the reagent liquid container.

In some implementations of this example, the sealed reagent liquid container may include a container body, a first membrane at one end of the container body facing the collection pad, and a second membrane at another end of the container body facing the internal protrusion, with the system configured such that depression of the cap onto the liquid collection device dislodges the first and second membranes.

In some implementations of this example, the sealed reagent liquid container may instead be an ampule.

In another example, a liquid sample collection system may include a liquid collection device having a liquid collector, and a collection chamber, the liquid collector configured to receive a liquid sample and to dispense the received liquid sample into the collection chamber; with the system configured such that the liquid collector is couplable and de-couplable from the collection chamber; and such that a sealing membrane seals the collection chamber; and such that the collection chamber holds a reagent; and such that the liquid collector is actuatable to disrupt the sealing membrane.

In some implementations of this example, the system may be configured such that actuation of the liquid collector to disrupt the sealing membrane takes the form of engaging or further engaging the liquid collector to the collection chamber.

In some implementations of this example, the liquid collector may have a liquid sample channel that is configured to pierce the sealing membrane when the liquid collector is engaged to or further engaged to the collection chamber.

In some implementations of this example, the liquid collector may additionally or instead have a cutting member configured to penetrate the sealing member along an arcuate path as the liquid collector is engaged to or further engaged to the collection chamber by rotating the liquid collector relative to the collection chamber.

In some implementations of this example, the cutting member may be configured to cut a hanging disk from the sealing member as the liquid collector rotates relative to the collection chamber.

In some implementations of this example, the sealing membrane may be sealed across an opening of the collection chamber.

In some implementations of this example, the system may instead include an adapter mounted to an opening of the collection chamber, with the sealing member sealed across an opening of the adapter.

In some implementations of this example, the liquid collector may further include a penetrating element that penetrates the sealing member and engages the liquid collector to the adapter such that removal of the liquid collector from the collection chamber also pulls the adapter out of the collection chamber.

In some implementations of this example, the penetrating element may include an expandable distal end that expands to engage the adapter after penetrating the sealing member.

In some implementations of this example, the liquid collector may include a lock that locks to the adapter as the liquid collector is engaged to or further engaged to the collection chamber such that disengagement of the liquid collector from the collection chamber also disengages the adapter from the liquid collection chamber.

In some implementations of this example, the lock may be part of a ratchet mechanism.

In some implementations of this example, actuation of the liquid collector may take the form of depressing a portion of the liquid collector relative to the collection chamber.

In some implementations of this example, the portion of the liquid collector may take the form of a plunger that is translatable relative to a body of the liquid collector.

In some implementations of this example, the plunger may include a fluid path for the collected sample extending from a proximal end of the plunger to a distal end of the plunger.

In some implementations of this example, the proximal end of the plunger may be in fluid communication with a collection pad configured to collect the collected sample.

In some implementations of this example, the plunger and the body may include a seal extending between the plunger and an interior wall of the body.

In some implementations of this example, the liquid collector may include a cap translatable relative to the body, and the plunger and the cap may further include a seal extending between the plunger and an interior wall of the cap.

In some implementations of this example, the body may include a liquid chamber proximal to the plunger, with the system configured such that collected sample in the liquid chamber of the body causes the plunger to translate distally to disrupt the membrane.

In some implementations of this example, the body includes a flow channel along an exterior of the plunger that opens when the plunger translates distally relative to the body.

In some implementations of this example, the liquid chamber of the body is fluidly connected to a reduced size fluid conduit extending proximally from the liquid chamber.

In some implementations of this example, the liquid collector has a body including a first body portion and a second body portion, with the system configured such that actuation of the liquid collector takes the form of depressing the first body portion relative to the second body portion from a first condition in which the sealing membrane is not disrupted and a second condition in which the sealing membrane is disrupted.

In some implementations of this example, the first and second body portions are rotationally fixed relative to one another at least when the first and second body portions are in the second condition.

In some implementations of this example, the first and second body portions are rotationally fixed relative to one another when the first and second body portions are in the first and second conditions.

In another example, a liquid sample collection system may include a liquid collection device with a liquid collector, and a collection chamber, the liquid collector configured to receive a liquid sample and to dispense the received liquid sample into the collection chamber; with the system configured such that the liquid collector is couplable and de-couplable from the collection chamber; with the system configured such that the liquid collector holds a reagent in a reagent cavity of the liquid collector in a sealed condition; and with the system configured such that the liquid collector is actuatable to unseal the reagent such that the reagent drains from the reagent cavity to the collection chamber.

In some implementations of this example, actuation of the liquid collector takes the form of coupling or further coupling the liquid collector to the collection chamber.

In some implementations of this example, the liquid collector includes a translatable body, with the system configured such that coupling or further coupling the liquid collector to the collection chamber translates the translatable body to open a drain and unseal the reagent cavity.

In some implementations of this example, coupling or further coupling the liquid collector to the collection chamber causes a proximal end of the collection chamber to push the translatable body in a proximal direction.

In some implementations of this example, the reagent cavity is defined by the translatable body and an additional interior surface of the liquid collector, and the liquid collector includes a first sliding seal between the translatable body and the additional interior surface, and the liquid collector includes a second sliding seal between the translatable body and an interior fluid channel of the liquid collector.

In some implementations of this example, the interior fluid channel includes a ramped surface; with the system configured such that coupling or further coupling the liquid collector to the collection chamber causes the ramped surface to unseal the second sliding seal such that the reagent drains from the reagent cavity to the collection chamber.

In some implementations of this example, coupling or further coupling the liquid collector to the collection chamber translates the translatable body to uncover the drain and unseal the reagent cavity.

In some implementations of this example, coupling or further coupling the liquid collector to the collection chamber pushes the translatable body past the drain.

In some implementations of this example, actuating the liquid collector to unseal the reagent such that the reagent drains from the reagent cavity takes the form of translating a first portion of the liquid collector relative to a second portion of the liquid collector.

In some implementations of this example, the liquid collector includes a plunger and a membrane, the membrane sealing the reagent cavity, with the system configured such that actuating the liquid collector causes the plunger to translate and disrupt the membrane such that the reagent can drain into the collection chamber.

In some implementations of this example, disrupting the membrane also opens a flow path for the liquid sample to flow into the collection chamber.

In some implementations of this example, translating a first portion of the liquid collector relative to a second portion of the liquid collector causes the liquid sample to flow into contact with the plunger such that the plunger translates distally to disrupt the membrane.

In some implementations of this example, translating the plunger distally opens a flow channel along an exterior of the plunger such that the liquid sample can flow past the plunger and into the collection chamber.

In some implementations of this example, actuating the liquid collector also opens a fluid path through the plunger for the collected liquid sample to flow into the collection chamber.

### BRIEF DESCRIPTION OF FIGURES

FIG. 1 shows a side plan transparent view of one example of a fluid collection device.

FIG. 2 shows a side plan transparent view of the fluid collection device of FIG. 1.

FIG. 3 shows a side cross-sectional view of another example of a fluid collection device, with the cap pushed down.

FIG. 4 shows an exploded view of the fluid collection device of FIG. 3.

FIG. 5 shows an example of a liquid sample collection system in which a reagent is maintained in a sealed condition in a cap of the device prior to use.

FIGS. 6 and 7 show additional example of liquid sample collection systems in which a reagent is maintained in a sealed condition in a cap of the device prior to use.

FIG. 8 shows an example of a liquid sample collection system in which a reagent is maintained in a sealed condition in a collection chamber of the device prior to use.

FIGS. 9-27 show additional examples of liquid sample collection systems in which a reagent is maintained in a sealed condition in a collection chamber of the device prior to use.

FIGS. 28-29 show an example of a liquid sample collection system in which a reagent is maintained in a sealed condition in a body of the device prior to use.

FIGS. 30-32 show additional examples of a liquid sample collection system in which a reagent is maintained in a sealed condition in a body of the device prior to use.

FIGS. 33-34 show an additional example of a liquid sample collection system in which a reagent is maintained in a sealed condition in a collection chamber of the device prior to use.

### DETAILED DESCRIPTION OF FIGURES

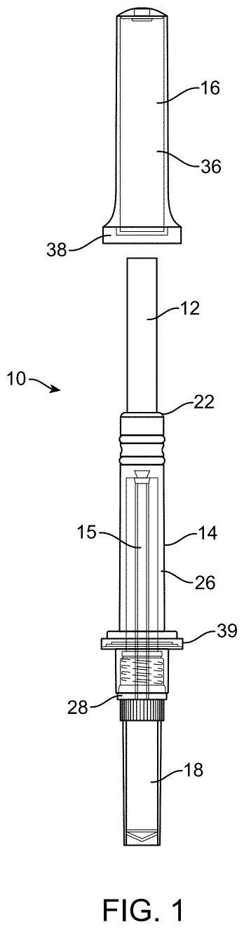

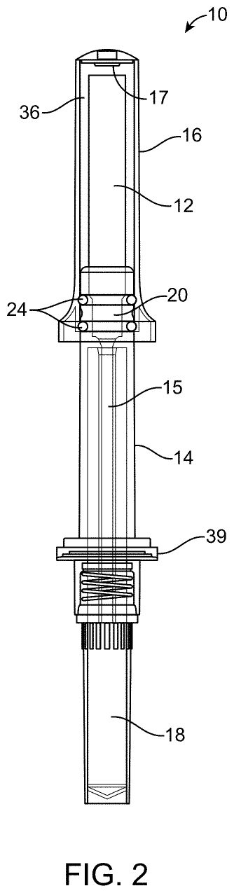

FIG. 1 shows an example of a liquid sample collection device 10 including a collection pad 12, a device body 14 that houses the collection pad, a cap 16 that provides a dual function of protection of the collection pad prior to use and compression of the fluid out of the pad, and a collection container 18. As shown by the cross section of FIG. 2, the device 10 may also be provided with a filter 20. These features are all incorporated into the single device 10, with the collection container 18 removably secured thereto. Additional features may also be incorporated into the device, as described herein. Material options for the collection pad 12 and filter 20 are described further below.

The device body 14 incorporates a collection pad 12 that is in fluid communication with the collection container 18. In a specific example, the collection pad 12 may be connected to one end of the device body 14 and the collection container 18 may be connected to the other end of the device body. Other connection options are possible, such as a direct connection or an indirect connection between the collection pad 12/device body 14 and/or the device body 14/collection container 18.

In the examples illustrated by FIGS. 1-4, the device body 14 has an upper open end 22 into which the collection pad 12 may be press fit or friction fit. In alternate options, the collection pad 12 may be glued, ultrasonically welded, laser welded, heat staked, or otherwise mechanically attached using any appropriate attachment system. If provided, the filter 20 is positioned below or otherwise downstream of the collection pad 12, such that compression of the collection pad 12 causes fluid contained in the collection pad 12 to be released from the pad 12 and to pass through the filter 20.

In one example, the filter 20 may have an outer diameter (OD) that closely matches the inner diameter (ID) of the upper open end 22 such that it is press fit into place. It is also possible for the OD of the upper open end 22 to have external grooves that can receive the one or more O-rings 24 for a liquid tight seal with the cap 16.

In the examples shown in FIGS. 1-4, a nozzle 15 is provided inside the device body 14, extending the length between the collection pad 12 and the collection container 18. The nozzle 15 can be a hollow tube that helps facilitate movement of the saliva to the collection container 18, after passing through the filter 20. The nozzle 15 effectively reduces the size of the cavity 26 (referenced in FIG. 1) between the collection pad 12 and the collection container 18. It is a thin hollow tube positioned within the cavity that has a smaller diameter than the diameter of the device body. The nozzle 15 allows more of the collected fluid to pass into the collection container 18 rather than getting stuck on sides of the device body during sample collection or transfer. This can be particularly useful for collection of smaller amounts of fluid, such as saliva. The nozzle 15 also extends down into the collection container 18 in order to help prevent leakage and/or splashing of the collected fluid near/around the connection between the device body 14 and the collection container 18. For example, if this connection is a threaded connection, the presence of nozzle 15 helps ensure that the collected saliva stays away from the threads when the container is opened. The nozzle 15 provides a pathway between the pad and the collection container. The nozzle can be connected to and a part of the device body 14. In the example shown by FIG. 2, the nozzle is positioned below the filter 20. In some implementations, a one-way valve connected to the patient-facing end of the nozzle to ensure that saliva passes into the collection pad and into the collection container, but not to flow in the opposite direction.

The collection container 18 is secured at a second end 28 of the device body 14. The collection container 18 may be secured to the device body 14 in any appropriate manner. Threaded connections are illustrated in the examples of FIGS. 1-4. In these examples, the collection container 18 has upper threading that is received by internal threading of the device body 14. It should also be understood than any other securement method may be used, such as press fit, clipping arms, magnetic attachment, or any other appropriate securement features. It should further be understood that the collection container 18 can be designed as an integral part of the device. For example, the collection container may be permanently connected to the device body. In this instance, the entire device 10 can be sent to a lab for analysis/testing of the sample, without the user having to remove the collection container. In any of the disclosed embodiments, the device (or the collection container on its own, if removable) may be inserted into an automation device as outlined below. If the collection container is not designed to be removable, it may be provided with a sample access point, such as an extraction port somewhere along the device. In a specific example, the extraction port may be positioned at the lower portion or bottom of the device, as described further herein.

A cap 16 is provided to cover and protect the collection pad 12 prior to use of the device 10. In use, the cap 16 is removed and fluid is collected. Once the fluid has been collected, the cap 16 is replaced over the collection pad 12, as shown in FIG. 2, and depressed. The cap 16 is shaped with a hollow channel 36 that fits over and receives the collection pad 12. As shown, the cap 16 may have a lower flange 38 that provides a ledge-like feature for a user's thumb or fingers to achieve leverage in order to depress the cap 16 in use. There may also be provided a compression feature 17 on the inside of the cap 16 at the top. Compression feature 17 allows further/maximum compression of the collection pad 12 inside the cap cavity 36 and device body 14.

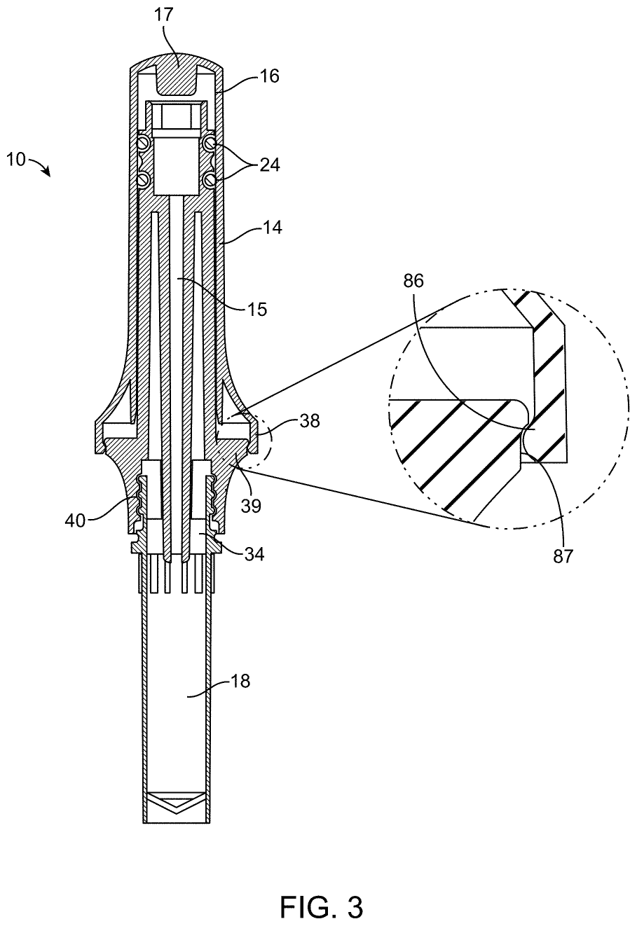

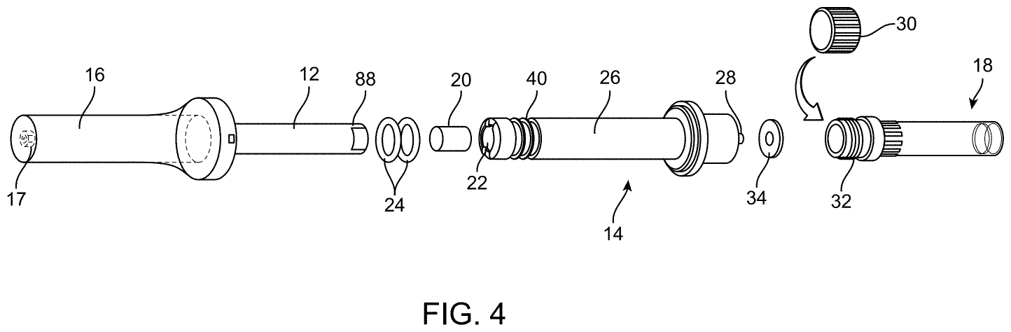

When the collection pad 12 is compressed via the cap 16, fluid contained therein is released from the collection pad 12, passes through the optional filter 20 if provided, and into the collection container 18. Because compression of the collection pad 12 takes place in the closed environment of the device 10, it may be helpful to provide a venting option to achieve a good compression. FIGS. 3 and 4 illustrate one example of a vent gasket 34, although any of the venting options described herein are possible and considered within the scope of this disclosure.

After collection of the fluid from the collection pad 12 takes place and is delivered to collection container 18, the collection container 18 may be removed from the device body 14, such that the fluid can be transported elsewhere for subsequent testing/processing. A collection container cap 30 (see FIG. 4) may be provided that can be secured to an upper open end 32 of the collection container 18 once it has been removed. The collection container cap 30 may be threadably secured to the upper open end 32, it may be a fliptop cap, or any other appropriate capping or sealing option. In alternate embodiments, the collection container may be a squeeze tube, similar to an eye dropper, that is squeezed to transfer liquid to a testing assay, such as a rapid diagnostic test.

Reference will now be made to specific features/options of the disclosed collection devices 10 of FIGS. 1-4.

### Collection Pad

The fluid that may be absorbed by collection pad 12 may be any appropriate fluid to be collected and/or tested. Non-limiting examples include but are not limited to oral fluid, saliva, urine, blood, diarrhea/feces, sweat, vaginal fluids, semen, or any other appropriate fluid. Although described in connection with healthcare, it should be understood that the disclosed device may be used for a myriad of other fluid collection uses. The collection pad may be associated with a sufficiency indicator that can change color or otherwise provide a visual indication that the collection pad is sufficiently wet with the fluid to be collected.

The collection pad is intended to absorb and hold liquid, but to also release the held liquid upon compression. The material of collection pad could comprise of fibers, foams or particulate materials made from polyesters, polyurethanes, polyethylene, polypropylene, PTFE, PVDF, elastomeric materials, thermoplastic polyurethane, thermoplastic polyethylene, nylon, cellulose, cellulose acetate, natural fibers, fabric, paper, polymer hydrogel (dry form and/or wet form) such as HEMA, NIPAM, etc., a natural or man-made material, sponge, cotton, or any combination thereof. The collection pad may be treated with dried buffer, saliva-production stimulants such as citrates, materials that prevent target analyte binding to the material, surfactants to help collect and release liquid, or any other appropriate material, or any combination thereof. It is also possible to provide a hollow collection pad, such that the collection pad is a tube that has a hollow tubular length.

### Cap

A cap 16 may be provided that can protect the collection pad 12 during transport and that can be pressed against the collection pad in order to force collected liquid sample out of the pad 12 and into the collection container 18. In some instances, the collected liquid sample travels through a filter on its way into the collection container 18. In a specific example, the cap 16 is designed and positioned such that it covers the collection pad 12. Pressure or force against the cap 16 can consequently apply pressure or force against the collection pad 12. This can squeeze the collected liquid through the filter (if provided) and into the collection container 18. In another specific example, the cap can form a seal with the outside of the collection device to ensure all fluid is directed through the collection pad (and optional filter) and into the collection container and prevent leaking of fluid to the outside of the device. For example, when the cap 16 is pressed, a seal between the O-rings 24 of the side of the device body 14 and the inner diameter of the cap 16 can create a liquid-tight seal.

In use, the cap 16 is pressed onto the device body 14 and pressure causes the liquid collected in the collection pad 12 to be transferred through the device body 14, through the nozzle 15 (if provided), through an optional filter 20, and into the collection container 18. O-rings 24 positioned around an upper portion of the device body 14 can help create a liquid tight seal when the cap 16 is pressed down. Pressure from compressed air that is pressed through the device body can be released through an optional vent. In one example, the vent may be threads between the device body 14 and the collection container 18. There may also be a feature 17 on the inside of the cap that adds extra compression to extract the maximum amount of liquid from the collection pad. This may be an interior plunger, spikes inside the cap, or any other appropriate feature. When the cap 16 reaches the end of its completed travel, detent features can fit over the device body 14 and engage to ensure a completed lock and snap into place. Detent features may be positioned at the end/bottom of the cap flange 38 and can snap over the body flange 39 just above where the collection container 18 is secured to the device 10.

The cap 16 and lower flange 38 may also be provided with feedback features that allow a user to know that the cap has been secured to the device body 14. Feedback features also indicate that the cap 16 has reached its full travel distance and that the swab has been fully compressed, signaling that no further action is needed and that all collected fluid available to transfer has been transferred to the collection container. In one example, the feedback features are provided as a detent/protrusion system that gives a tactile click feeling. This may be provided by a small internal protrusion 86 on the cap 16 and a similarly-shaped detent 87 on the ledge 38, which is illustrated by FIG. 3. It should also be understood that these components may be switched, such that the detent is on the cap and the protrusion is on the ledge/device body.

### Filter

The filter 20 is generally positioned below (or downstream of) the collection pad 12 and functions to filter out unwanted media from the liquid collected. The filter 20 can filter out undesired particles or other contaminants in the collected sample. Providing a filter 20 can help eliminate later centrifuging steps that would otherwise be required during testing of the collected sample. Providing a filter 20 can help concentrate the sample. Providing a filter 20 can help provide sample homogenization. Bio junks such as mucin clusters can be eliminated or grinded by passing through the filter 20, thus reducing the sample viscosity and facilitating liquid handling.

Non-limiting examples of particles that may be filtered out of the sample include but are not limited to molecular particulates, cells, proteins, cellular debris, nucleic acids, mucins, glycoproteins, bacteria, viruses, large biomolecule clusters, large size bacterial particles, dust particles, fibers, other environment contaminates, or any combination thereof.

Non-limiting examples of materials that may comprise the filter include but are not limited to fiber components, track-etched-membranes, and sintered particles that can offer various porosities or void volume to the filter. Porex, the current assignee of this application, manufactures and holds patent protection on various filters and/or filtration systems that may be usable in connection with this disclosure. The filter may be used to mechanically and/or chemically filter out any of the above-described particles from the liquid sample. It is possible to incorporate reagents, additive, or buffers into the filter that can be mixed with the collected fluid as it passes through. Such reagents, additives, or buffers can be added in order stabilize the liquid sample/saliva or analytes and/or to allow more accurate detection and/or to provide for viral inactivation, viral lysis, to stop or prevent bacterial growth, or any other appropriate reason for use of reagents, additives, and/or buffers.

### Collection Container

There is a tight seal made when the collection container 18 is screwed (or otherwise joined) onto the device body 14. The collection container 18 may be any type of container that can collect, hold, and/or transport a liquid sample. Embodiments include but are not limited to vials, centrifuge tubes, cryo vials, blood collection tubes, saliva collection tubes, syringes, dropper bottles with a lower dispenser tip or dropper that allows the sample to be squeezed out of the collection container once collected (and filtered, etc.), syringes, or any combination thereof.

The collection container 18 may be connected to the device body 14 in any appropriate manner. Non-limiting examples of various joining mechanisms include but are not limited to a threaded connection, a press fit connection, a cone-in-cone friction fit connection, a magnetic connection, side clip arms or fastener connection, or any other appropriate type of joining mechanism. In some implementations, the collection container may be made integral with the device body, such that the entire device is sent out for testing. FIGS. 3-4 illustrate a collection container 18 that has upper threading 32 (shown in FIG. 4). Upper threading 32 may cooperate with lower threading 40 of the device body (shown in FIG. 3).

Mixing Collected Fluid with Reagent

In some implementations it may be desirable to mix the collected fluid in the device with a reagent, stabilizer, buffer, and/or additive (for brevity, all of which are referred to as a “reagent” in this patent). The reagent may be mixed with the collected fluid for sample preservation, diagnosis, or other reasons. Prior to use, the reagent may be maintained in a sealed condition in the device cap, body, and/or collection chamber, with the sealing preserving the reagent in an air tight or air free condition prior to use of the device.

### Reagent in Cap

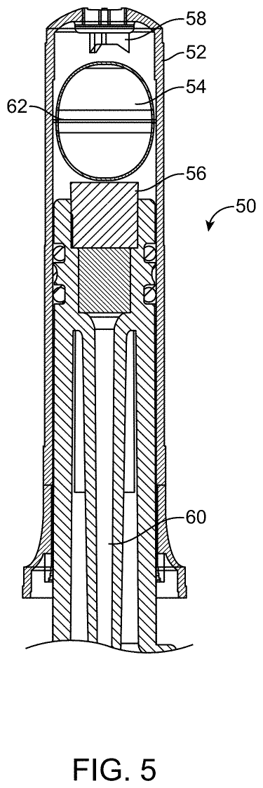

FIG. 5 shows an example of a liquid sample collection system including a liquid collection device 50, a cap 52 for the liquid collection device, and a sealed reagent liquid container 54 installed in the cap. Depression of the cap 52 relative to the liquid collection device 50 unseals the reagent liquid container 54.

FIG. 5 shows the cap 52 partially depressed relative to the liquid collection device 50. As the cap 52 is further depressed, the sealed reagent liquid container 54 will be compressed between the collection pad 56 (which itself collapses as the cap 52 is depressed) and a sharp protrusion 58 extending downwardly from the top interior of the cap 52. As the cap 52 is further depressed, the protrusion 58 will pierce and disrupt the reagent liquid container 54, causing a reagent liquid held in the container to flow down through the collection pad 56, through passageway 60, and into a collection chamber (not shown).

In the particular example of FIG. 5 the sealed reagent liquid container 54 is an ampule made from a pierceable membrane such as a polyvinyl alcohol film (PVA), similar to ampules used as detergent pods. In other examples, the ampule may be a capsule, sealed cup, or other container that is configured to hold the reagent in a sealed condition until the cap 52 is depressed relative to the liquid collection device 50.

In the particular example of FIG. 5 the sealed reagent liquid container 54 includes a flange 62 that provides a friction fit inside the cap 52, retaining the sealed reagent liquid container 54 inside the cap 52. In other implementations the sealed reagent liquid container 54 may be retained in position in the cap 52 in other ways.

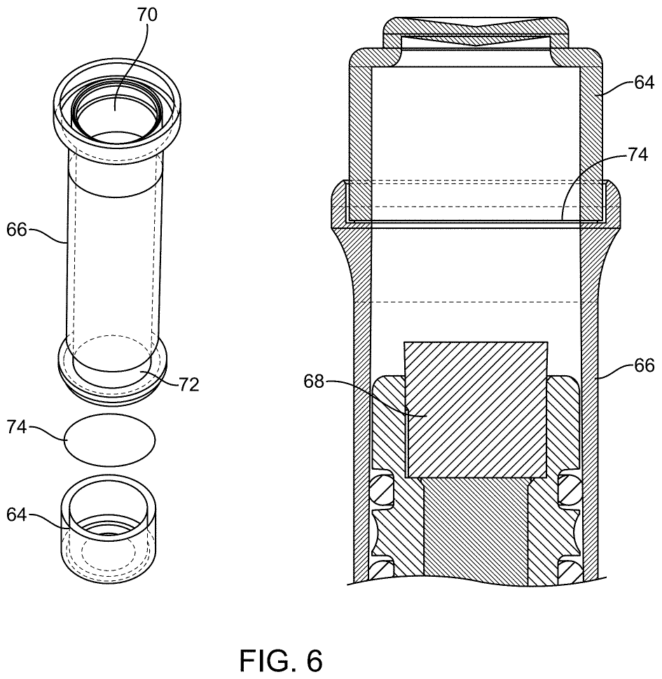

FIG. 6 shows another example of a liquid sample collection system with a sealed reagent liquid container 64 installed in a cap 66. As in the previous example, depression of the cap 66 relative to the liquid collection device unseals the reagent liquid container 64 to cause a reagent liquid to flow through a collection pad 68 and into a collection chamber (not shown in FIG. 6).

In this example, the cap 66 has a first open end 70 that receives the liquid collection device and a second open end 72 that connects to the sealed reagent container 64. The sealed reagent container 64 includes a membrane 74 that will be dislodged by the collection pad 68 (e.g. by shearing under compression) during depression of the cap 66 relative to the liquid collection device, thereby allowing the liquid reagent to flow out of the container 64. The reagent container 64 may be connected to the cap 66 by heat welding, threading, adhesive, snap fit, ultrasonic welding, or any other manner that provides a liquid tight seal between the container 64 and the cap 66. The membrane 74 may be connected to the reagent container 64 by heat welding, adhesive, ultrasonic welding, or any other suitable manner to form a liquid tight sub-assembly.

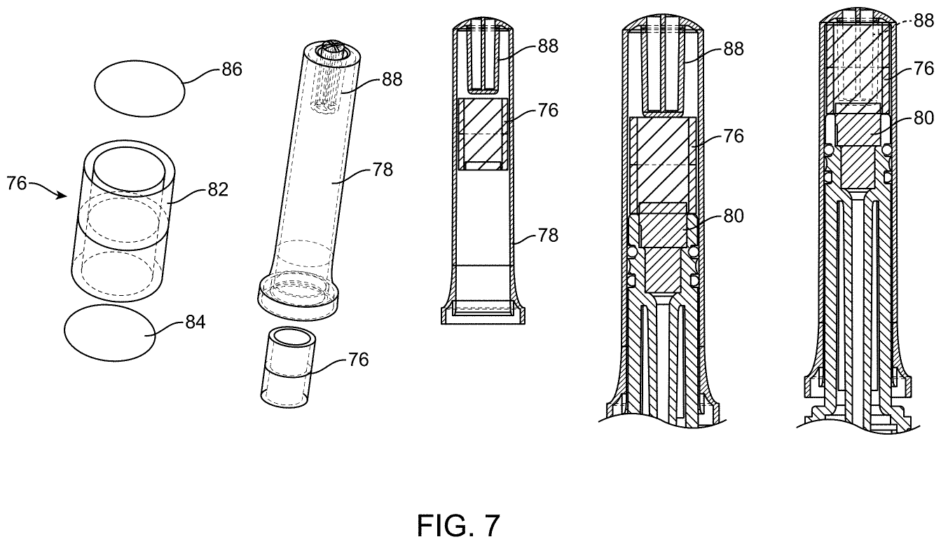

FIG. 7 shows another example of a liquid sample collection system with a sealed reagent liquid container 76 installed in a cap 78. As in the previous example, depression of the cap 78 relative to the liquid collection device unseals the reagent liquid container 76 to cause a reagent liquid to flow through a collection pad 80 and into a collection chamber (not shown in FIG. 7).

In the example of FIG. 7 the sealed reagent container 76 includes a container body 82, a first membrane 84 at one end of the container body 82 facing the collection pad 80, and a second membrane 86 at the other end of the container body 82 facing the top of the cap 78. The container body 82 may be configured to be held temporarily in position inside the cap 78 by a friction fit. The top of the cap 78 includes a downwardly extending protrusion 88. As shown in FIG. 7 depression of the cap 78 dislodges both membranes 84, 86, with the collection pad 80 dislodging the first membrane 84 and the protrusion 88 dislodging the second membrane 86. In some implementations the protrusion 88 may include sharpened portions to facilitate puncturing or otherwise dislodging the membrane 86.

As shown in FIG. 7 depression of the cap 78 causes the reagent container 76 to vertically translate inside the cap 78. In some implementations, the protrusion 88 may be configured to function like a plunger and push the liquid reagent out of the container body 82 and down through the collection pad 80, leaving no dead volume inside the container body 82.

In another example (not shown in the figures) the cap of the device may further include a disruptable membrane defining a chamber in the cap 16 for holding a reagent in a sealed condition. For example, the reagent container 64 and cap 66 shown in FIG. 6 could be replaced with a one-piece cap that includes a membrane (similarly secured and positioned to the membrane 74 shown in FIG. 6) such that one area of the cap is sealed off and can hold a reagent in a sealed condition until it is disputed by depressing the cap. In other words, in this example, the cap in combination with the membrane may define a sealed reagent liquid container installed in the cap.

### Reagent in Collection Chamber

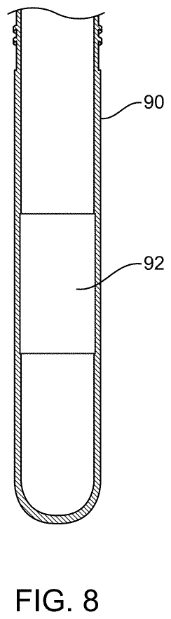

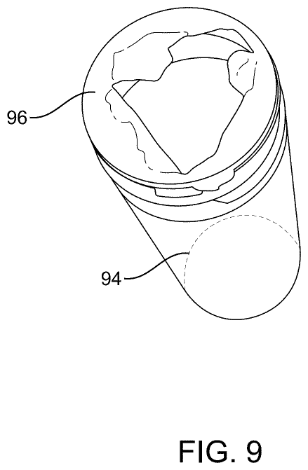

FIG. 8 shows an example of a liquid sample collection system in which the reagent is held in a sealed condition inside the collection chamber 90. In this example the liquid reagent is held inside a dissolvable ampule 92 located inside of the collection chamber 90. In this example the ampule is of a material that will dissolve when contacted by saliva or other liquid being collected by the system but does not dissolve when contacted by the reagent held inside the ampule 92.

In FIG. 8, only the collection chamber 90 and ampule 92 is shown, it being understood that the other components of the system may be similar to those of the system shown in FIG. 1.

In other examples, instead of a sealed ampule in the collection chamber, the collection chamber itself may be sealed to maintain a reagent in a sealed condition prior to use. For instance, a liquid sample collection system may include a liquid collector (e.g. the same as or similar to the liquid collector including body 14 in FIG. 1) and a collection chamber (e.g. the same as or similar to the collection chamber 18 in FIG. 1) in which the liquid collector is couplable and de-couplable from the collection chamber and in which coupling (or further coupling) the liquid collector to the collection chamber disrupts a sealing membrane on the collection chamber and unsealing the liquid reagent held in the collection chamber prior to introduction of the collected liquid sample into the collection chamber. For instance, the liquid collector and the collection chamber may be threaded such that the liquid collector can be screwed onto the collection chamber, with the act of screwing the liquid collector onto the collection chamber causing the disruption of the sealing membrane on the collection chamber.

FIG. 9 illustrates an example of a collection chamber 94 including a sealing membrane 96 that has been disrupted to unseal the collection chamber 94. The sealing membrane 96 may be a plastic, a metal foil, a polyethylene lined metal foil, or any appropriate material that may be secured to the rim of and across the opening of the collection chamber 94 to preserve the liquid reagent held within the collection chamber 94. In some implementations, air may be removed from the collection chamber as it is sealed with the liquid reagent inside. Forming a vacuum in the collection chamber as it is sealed may avoid the need to include venting in the system.

In some implementations the liquid collector and collection chamber may be supplied in a partially disengaged or completely disengaged condition (e.g. may be provided as two disassembled pieces) such that engaging or further engaging the liquid collector to the collection chamber (e.g. screwing the liquid collector onto the collection chamber) results in the sealing membrane 96 on the collection chamber 94 being cut, pierced, or otherwise disrupted.

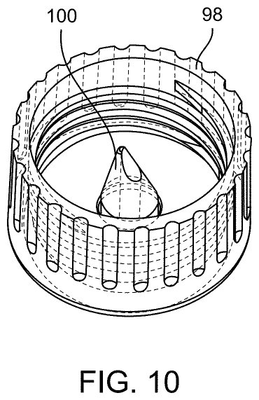

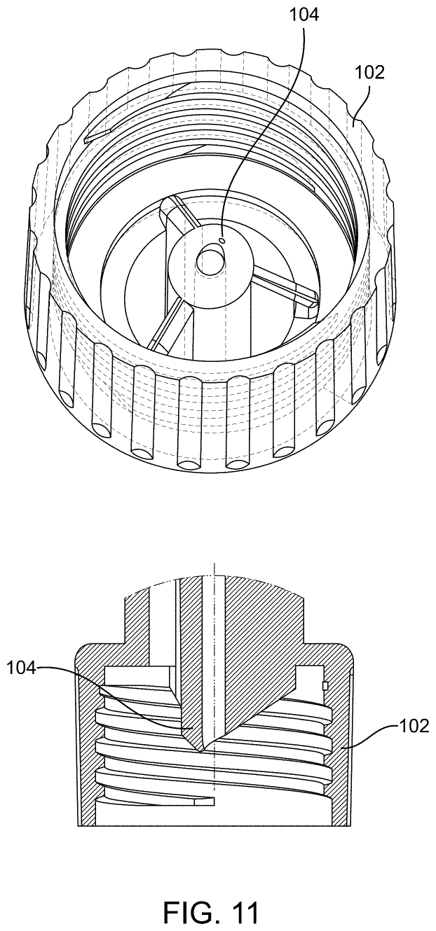

In one example, the liquid collector includes a liquid sample channel (e.g. the same as or similar to nozzle 15 in FIG. 1) with a distal end that is positioned and configured to disrupt the sealing membrane on the collection chamber when the liquid collector is assembled to and engaged with the collection chamber. FIG. 10 shows one example of a distal end of a liquid collector 98 with a distal end of a liquid sample channel 100 that is positioned and configured to pierce or spear a sealing membrane on a collection chamber when the liquid collector is assembled to and engaged with the collection chamber. FIG. 11 shows another example of a distal end of a liquid collector 102 including a sample channel 104 and ribs extending from the sample channel 104 that are positioned and configured to pierce or spear a sealing membrane on a collection chamber when the liquid collector is assembled to and engaged with the collection chamber. In this example, the ribs are positioned to create a larger disruption/opening in the membrane, which may in some instances make it easier to dispense a liquid into the collection chamber. In some instances, forming a larger disruption/opening in the membrane may also ease downstream lab work with the collection chamber (e.g. making it easier to withdraw liquid from the collection chamber).

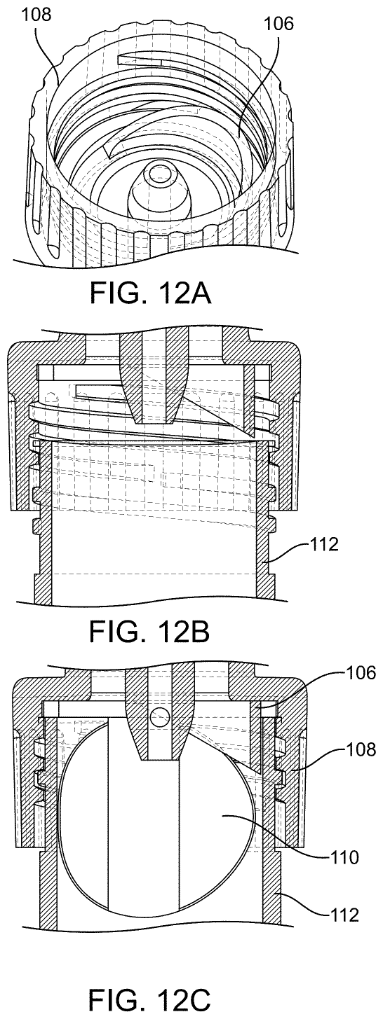

In other implementations, the liquid collector may include a cutting member that is configured to penetrate the sealing member of the collection chamber along an arcuate path as the liquid collector is engaged to or further engaged to the collection chamber by rotating the liquid collector relative to the collection chamber. FIGS. 12 a-c show one example of this, with a curved cutting member 106 incorporated into the distal end of the liquid collector 108. As shown in FIGS. 12 b-c, the cutting member 106 is configured to cut a disk 110 from the sealing membrane that hangs down after the liquid collector 108 has been threaded onto the collection chamber 112. The cutting member 106 may be sized and configured such that it cuts less than a complete 360 degree path through the sealing membrane to leave the hanging disk connected to the rest of the sealing membrane by a small portion.

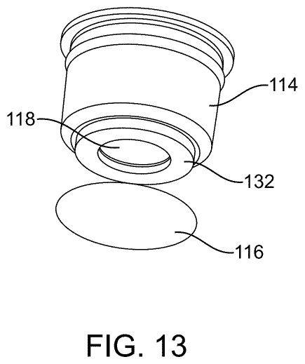

The example systems of FIGS. 9-12 include a sealed collection chamber in which the sealing membrane is sealed directly to the rim of the collection chamber's opening. In other implementations the sealing membrane may be sealed onto an adapter that is inserted into the collection chamber's opening to seal the collection chamber and preserve until use a reagent liquid held in the collection chamber. FIGS. 13-21 show examples of systems using adapters including sealing membranes.

FIG. 13 includes one example of an adapter 114 and sealing membrane 116, shown in disassembled condition. The adapter 114 and sealing membrane 116 are configured for the sealing membrane 116 to be sealed across an opening 118 in the adapter 114. The sealing membrane 116 may be a metal foil, a plastic film, or any material that can seal a collection chamber and preserve a reagent held within the collection chamber. The sealing membrane 116 may be adhered, welded, or otherwise mounted and secured to the adapter 114.

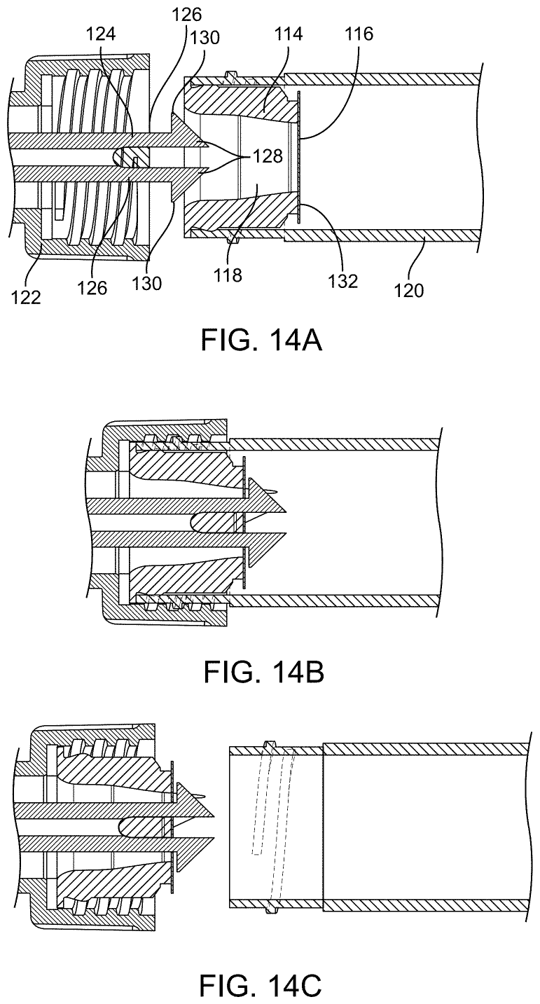

FIG. 14(a) shows the sealing membrane 116 sealed across the opening 118 in adapter 114, with the adapter 114 mounted to an opening of a collection chamber 120. FIG. 14(a) also shows a distal end of a liquid collector 122 including a penetrating element 124. As shown in FIGS. 14(b) and (c), the penetrating element 124 is configured to penetrate the sealing membrane 116 and engage the liquid collector 122 to the adapter 114 such that removal of the liquid collector 122 from the collection chamber 120 also pulls the adapter 114 out of the collection chamber 120. In this particular example the penetrating element 124 is a pair of resilient arms 126, each of which has a piercing tip 128 for piercing the sealing membrane 116 and a shoulder 130 configured to engage an inner rim 132 of the adapter 114.

In use, as the liquid collector 133 is engaged to the collection chamber 120 (e.g. by threading the liquid collector 133 onto the collection chamber 120) the walls of the opening 118 in adapter 114 will force the resilient arms 126 towards one another as the arms 126 move through the opening 118 of the adapter 114 until the piercing tips 128 pierce the sealing membrane 116 and the shoulders 130 pass the inner rim 132 of the adapter 114, allowing the resilient arms 126 to spring back outwards such that the shoulders 130 are in an abutting relationship with the inner rim 132. In this manner, when the liquid collector 122 is eventually removed from the collection chamber 120 (e.g. to prepare the collection chamber 120 to be capped for shipping to an analysis site or being eventually removed at the analysis site) the adapter 114 will be automatically removed from the collection chamber 120 as the liquid collector 122 is removed from the collection chamber 120.

FIGS. 13-14 illustrated one example of a liquid sample collection system where the liquid collector includes a locking mechanism that locks it to an adapter associated with the collection chamber such that disengagement of the liquid collector from the collection chamber also disengages the adapter from the collection chamber. FIGS. 15-16, FIGS. 17-19, and FIGS. 20-1 illustrate additional examples of this. All of these examples facilitate use of standard lab vials in the liquid sample collection system without leaving any membrane on the vial, which may facilitate downstream lab processing including automated lab processing in which membrane remainders could negatively impact.

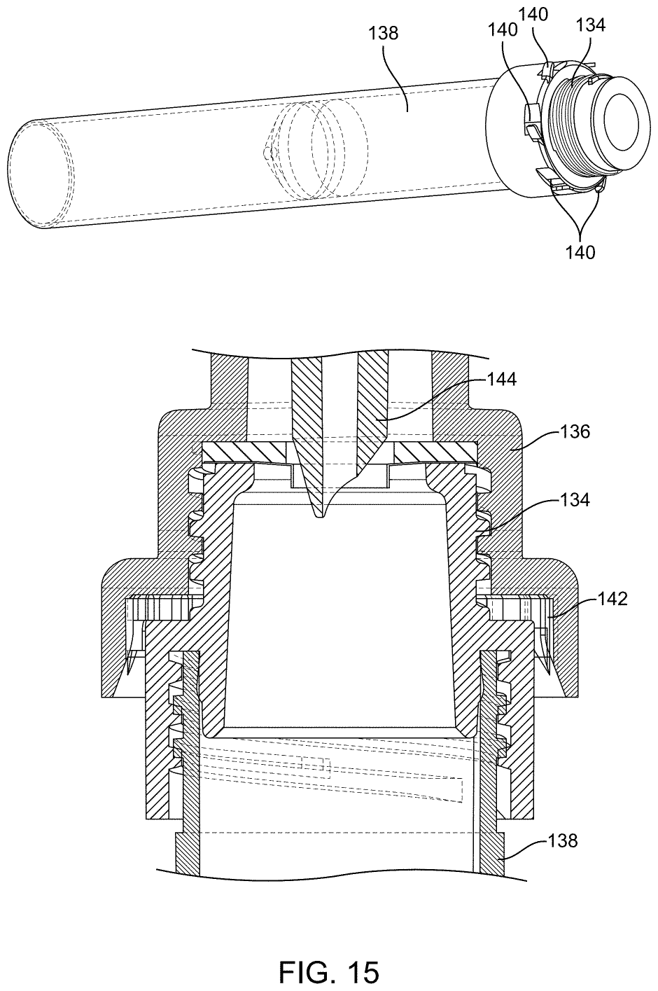

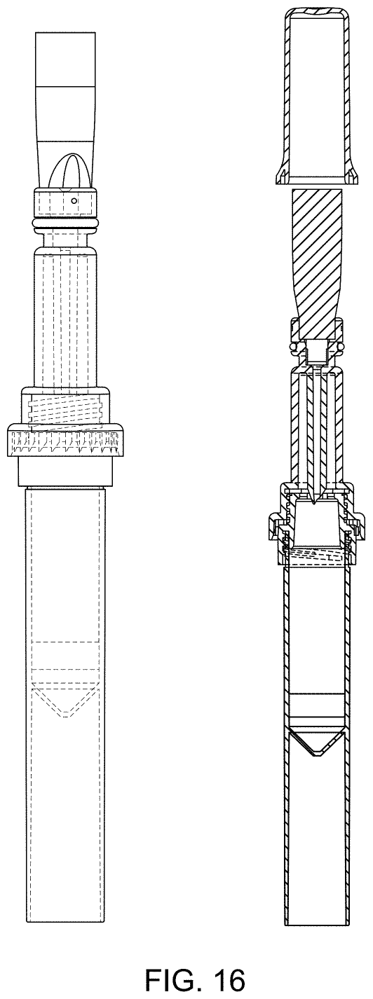

FIGS. 15-16 illustrate an example of a liquid sample collection system incorporating a ratchet mechanism for locking an adapter 134 to a liquid collector 136 when the liquid collector 136 is engaged to a collection chamber 138. In this particular example, the lock is a ratchet mechanism including pawls 140 on the adapter 134 that engage teeth 142 on the liquid collector 136 that allow the liquid collector 136 to be screwed onto the adapter 134 but not unscrewed from the adapter 134 such that unscrewing of the liquid collector 136 will also remove the adapter 134. As in the previous example, engaging the liquid collector 136 to the collection chamber 138 will pierce a sealing membrane associated with the adapter 134 (in this example, piercing by a distal end of liquid sample channel 144).

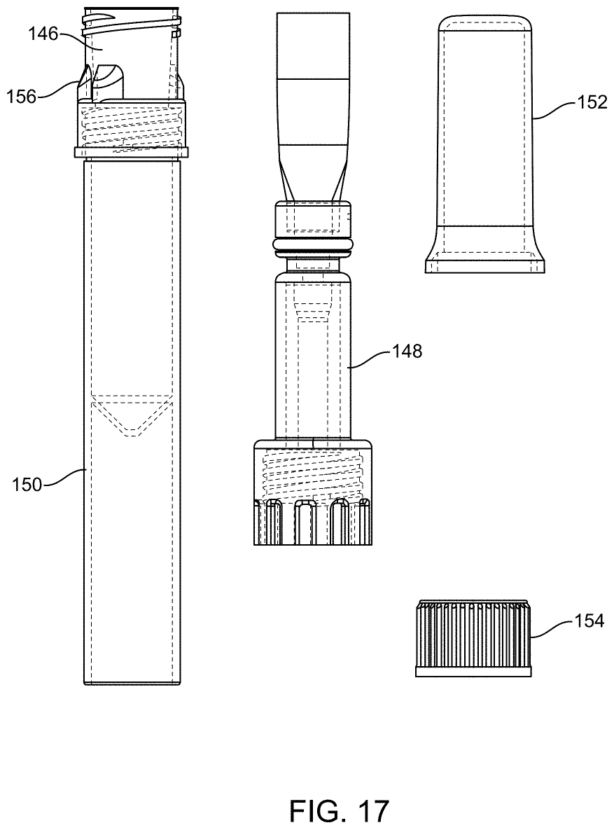

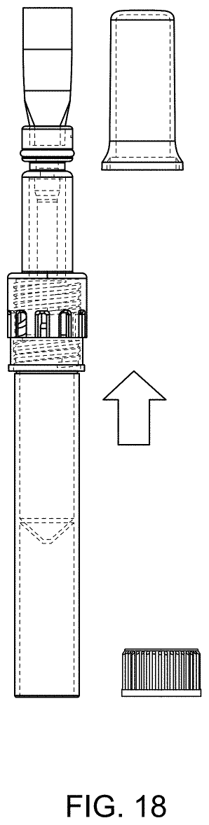

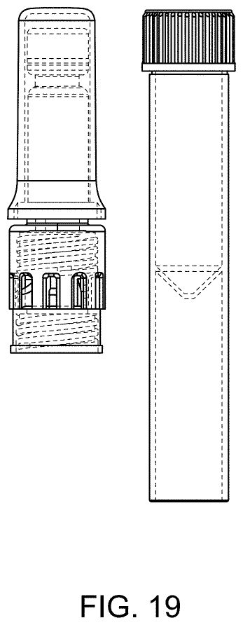

FIGS. 17-19 illustrate another example of a liquid sample collection system incorporating a ratchet mechanism for locking an adapter 146 to a liquid collector 148 when the liquid collector 148 is engaged to a collection chamber 150. FIG. 17 shows the liquid sample collection system as a kit in the form that it would be supplied to a user. The kit of FIG. 17 includes the collection chamber 150 with the adapter 146 and its membrane pre-installed on the collection chamber 150 to seal and preserve a reagent (not shown) inside the collection chamber 150. The kit also includes the liquid collector 148 provided unattached to the collection chamber 150, as well as a liquid collector cap 152 and a vial cap 154. FIG. 18 shows the liquid collector 148 engaged with the collection chamber 150. During engagement, the ratchet mechanism will lock the liquid collector 148 to the adapter 146 as the membrane on the adapter 146 is pierced. After use (which may be in a manner similar to that discussed above for the example of FIGS. 1-4) the liquid collector 148 is disengaged from the collection chamber 150. During disengagement the adapter 146 will remain locked to and removed with the liquid collector 148, such that as shown in FIG. 19 the collection chamber 150 will no longer be associated with the adapter 146 and can be sealed with the standard vial cap 154 for returning to a lab or other processing site. The liquid collector 148 and liquid collector cap 150 may be disposed of after use.

In the example of FIGS. 17-19, pawls 156 on the adapter 146 interact with teeth on the liquid collector 148 to lock the liquid collector 148 to the adapter 146 during engagement of the liquid collector 148 to the collection chamber 150.

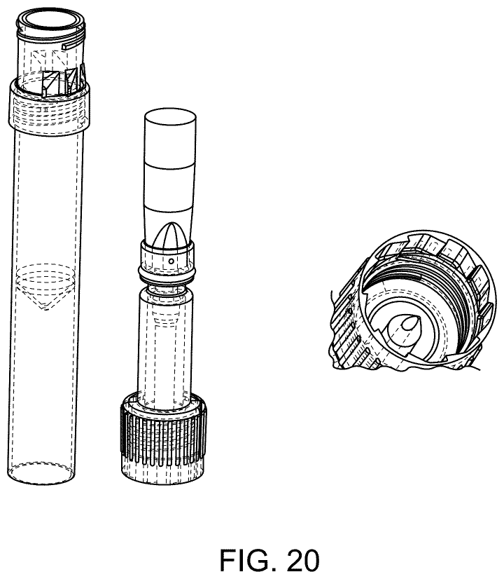

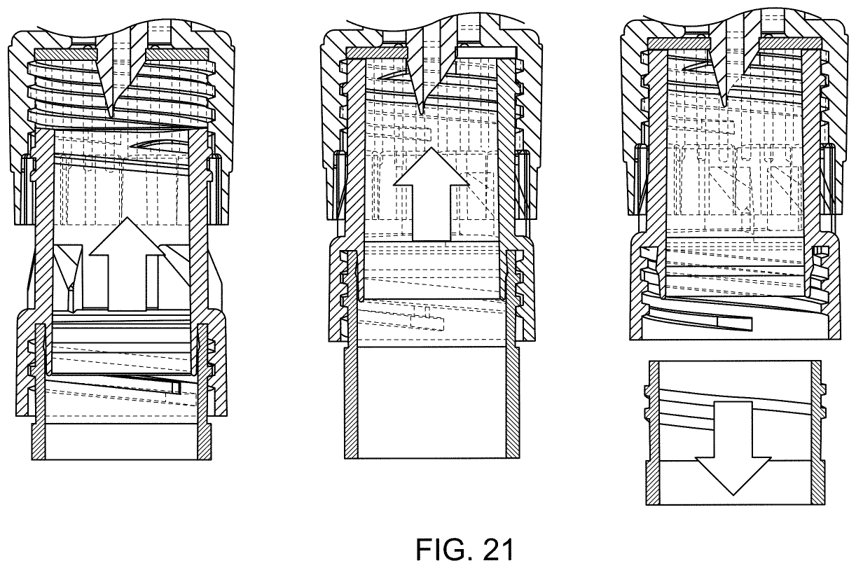

FIGS. 20-21 show one more example of a liquid sample collection system incorporating a ratchet mechanism such that an adapter originally supplied on the collection chamber will be locked to and removed with the liquid collector.

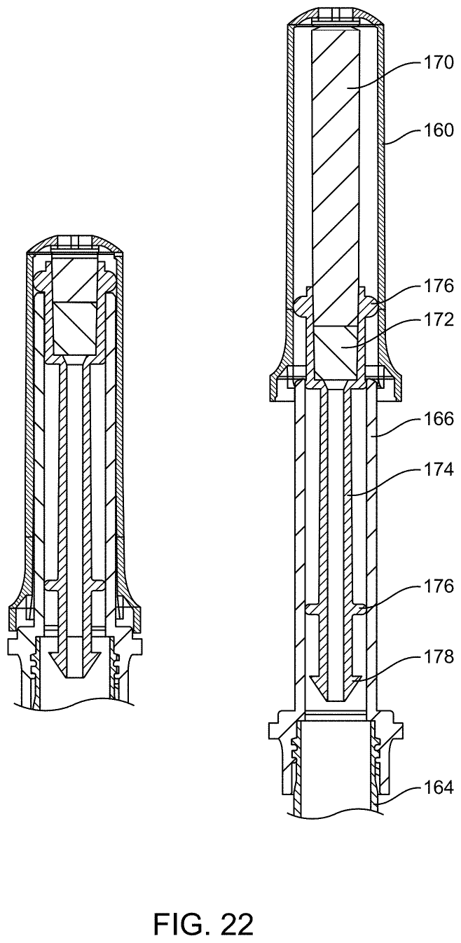

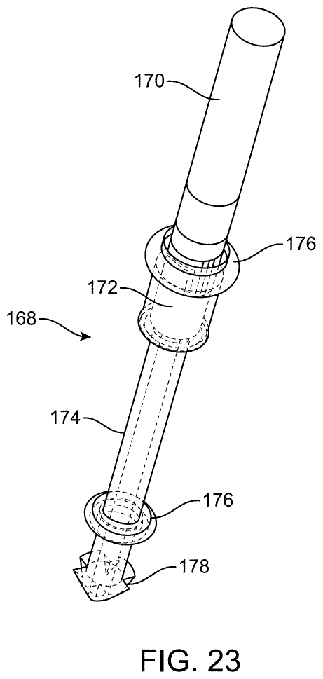

FIGS. 22 and 23 show another example of a liquid sample collection system. In this example, depressing the liquid collector (more specifically in this example, depressing the cap 160 of the liquid collector) will disrupt a sealing membrane on the collection chamber 164. The liquid collector in this example includes a body 166 and a translatable assembly 168 that is slidably mounted in the body and that will translate in the body when the cap 160 is depressed. The translatable assembly 168 includes a collection pad 170, a filter 172, and a plunger body 174. The plunger body 174 include sliding seals 176 that form fluid tight, but slidable, seals relative to the interior of the cap 160 and the body 166.

The plunger body 174 also includes a distal end 178 that is configured to pierce the sealing membrane such that, after depressing the translatable assembly 168, a fluid path will be formed from the collection pad 170, through a channel in the plunger body 174, to the collection chamber 164.

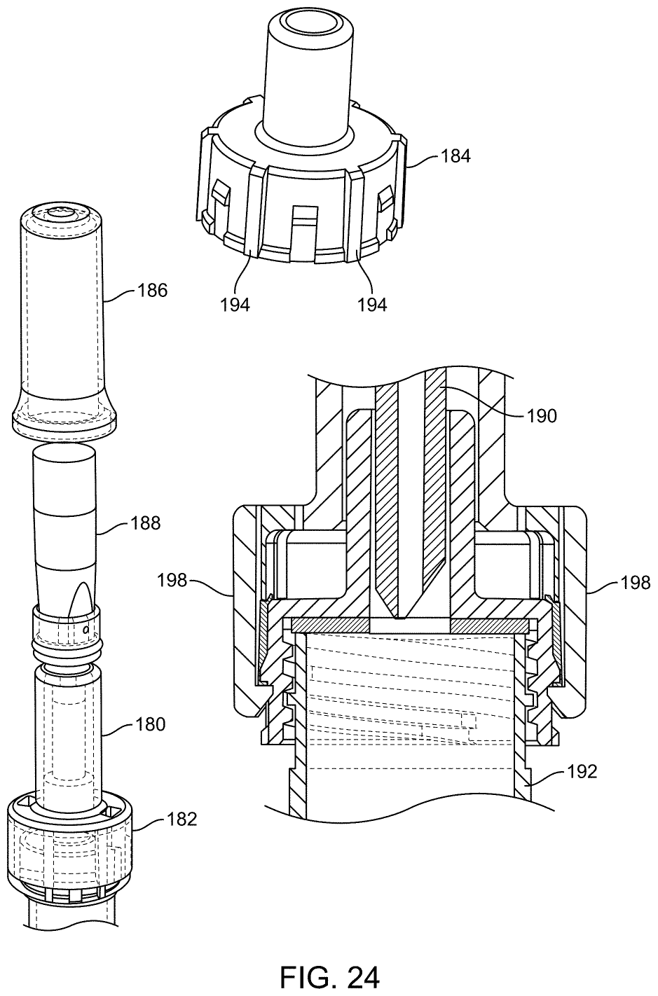

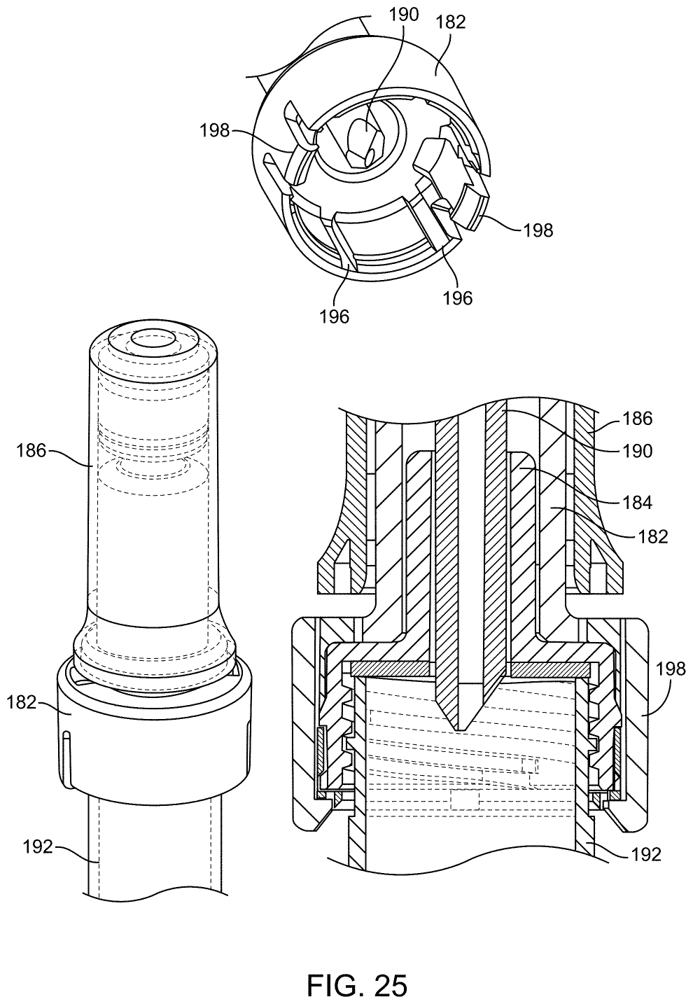

FIGS. 24 and 25 show another example of a liquid sample collection system. In this example, the liquid collector 180 has a body with a first body portion 182 that is actuated be depressing it relative to a second body portion 184 from a first condition (shown in FIG. 24) in which the sealing membrane is not disrupted to a second condition (shown in FIG. 25) in which the sealing membrane is disrupted.

In this example, the first body portion 182 is slidably mounted on the second body portion 184 such that depression of the cap 186 and compression of the collection pad 188 causes the first body portion 182 to slide downwardly on the second body portion 184 such that the distal end of the liquid sample channel 190 of the first body portion 182 will pierce a sealing membrane extending across the opening of the collection chamber 192.

Ribs 194 on the exterior of the second body portion 184 interact with grooves 196 in the first body portion 182 such that the first and second body portions are allowed to slide relative to one another while remaining rotationally fixed relative to one another. Once the first body portion 182 has been slid to the second condition (shown in FIG. 25), resilient arms 198 on the first body portion 182 will latch onto the second body portion 184 such that it cannot be slid back to the first condition, and such that rotation of the first body portion 182 will also rotate second body portion 184 to unthread it from the collection chamber 192, allowing the liquid collector to be removed from the collection chamber 192 as a single unit.

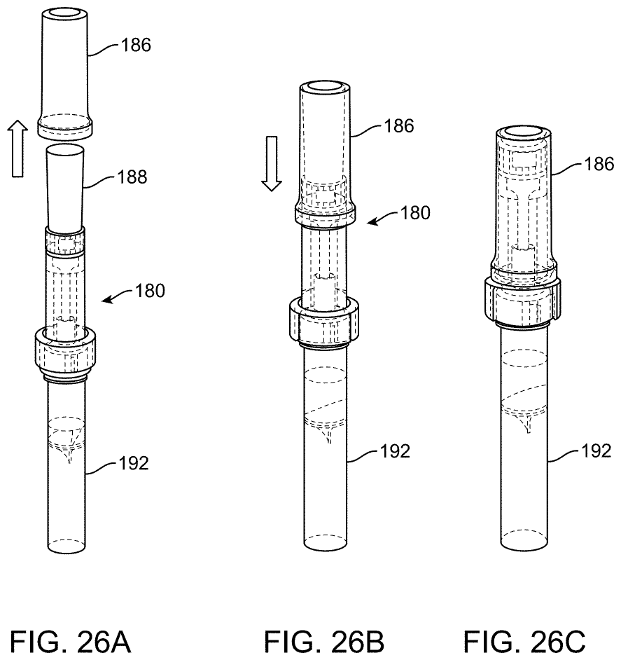

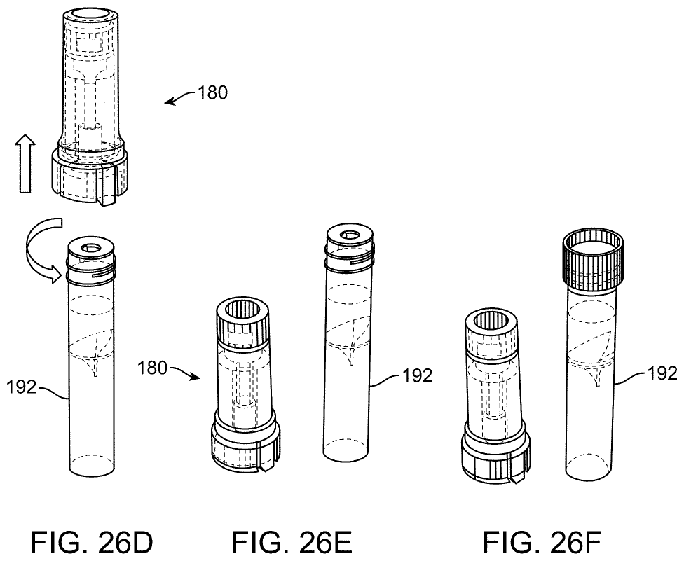

FIGS. 26 (a)-(e) show a method of using the liquid sample collection system of FIGS. 24-25. In FIG. 26 (a) the cap 186 is removed and a liquid sample is collection in the collection pad 188. In FIGS. 26 (b) and (c) the cap 186 is replaced and pressed downwardly, resulting in: (1) the first body portion 182 sliding downwardly on the second body portion 184 such that the liquid sample channel 190 disrupts a sealing membrane on the collection chamber 192, (2) the first body portion 182 latches to the second body portion 184 in this slid down position, and (3) the collection pad 188 is compressed by the cap 186 causing the collected liquid sample to pass through the channel 190 into the collection chamber 192 where it mixes with a reagent. In FIGS. 26 (d) and (e) the liquid collector 180 is removed as a unit from the collection chamber 192. In FIG. 26 (f) the collection chamber 192 is capped such that it is ready for transport to a diagnostic facility for analysis.

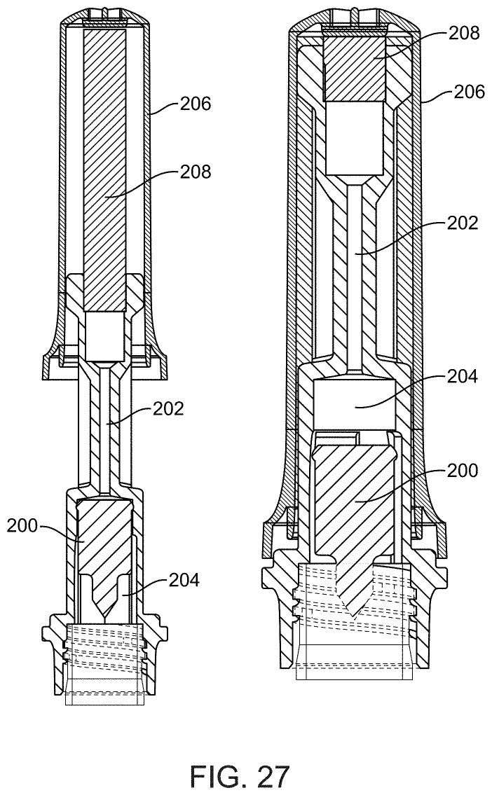

FIG. 27 shows an example of a liquid sample collection system including a translatable plunger 200 in the liquid collector that is configured to pierce a sealing membrane on the collection chamber. In this example, the liquid sample channel includes a narrower portion 202 and a wider portion 204, with the plunger 200 housed in the wider portion. When the cap 206 is depressed and the collection pad 208 is compressed, the collected liquid sample will flow through the narrower portion 202 of liquid sample channel and exert pressure on the plunger 200, forcing the plunger 200 to translate downwardly in the wider portion 204 of liquid sample channel until it disrupts the membrane on the collection chamber and allows the collected liquid sample to flow around the plunger 200 and into the collection chamber. As shown in this particular example, the wider portion 204 of the liquid sample channel may be slightly narrower at its proximal end (to hold the plunger 200 in place by friction until it is displaced by the collected liquid sample) and wider at its distal end (to facilitate the plunger fully descending in use and to provide sufficient room around the plunger for the collected liquid sample to flow into the collection chamber).

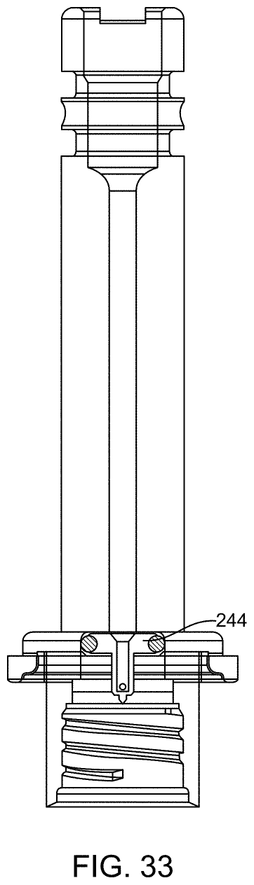

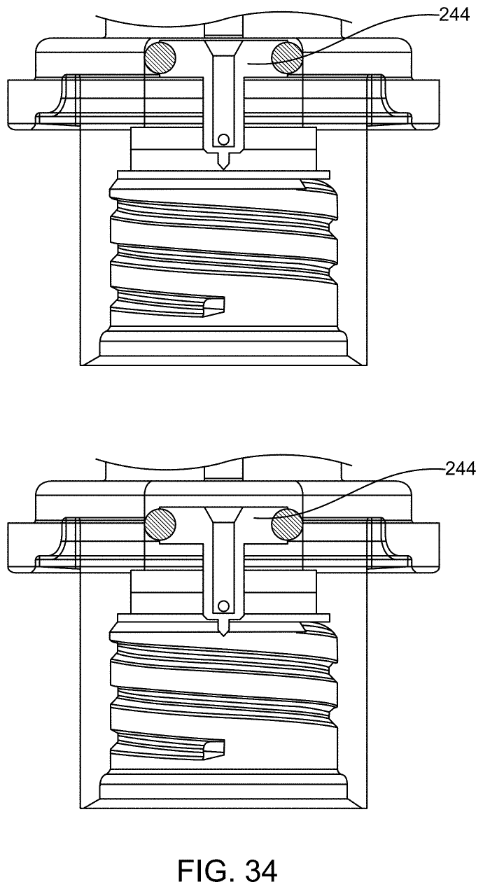

In the example of FIG. 27, the liquid sample flows around the plunger 200 after it has translated downwardly to disrupt the membrane. In other examples, including the example shown in FIGS. 33-34, the plunger 244 may have an internal fluid passageway through which the liquid sample flows after the plunger 244 has translated downwardly to disrupt the membrane.

### Reagent in Liquid Collector Body

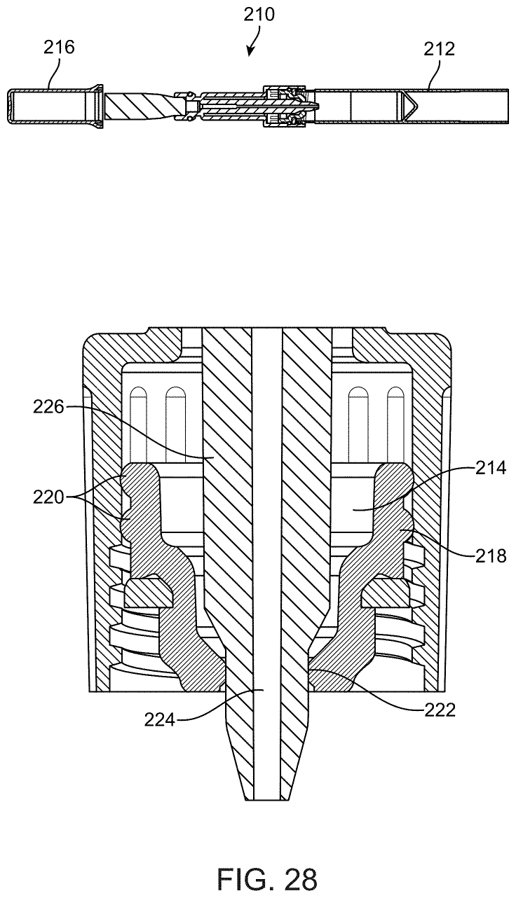

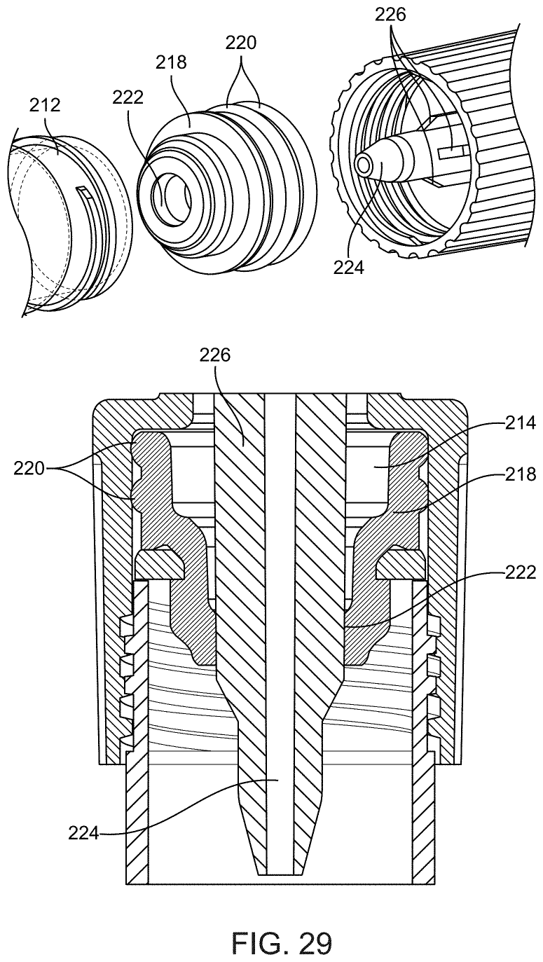

FIGS. 28-29 show an example of a liquid sample collection system where the reagent is maintained in a sealed, preserved condition in the body of the liquid collector prior to use. The system in FIGS. 28-29 includes a liquid collector 210 and a collection chamber 212, the liquid collector 210 configured to receive a liquid sample and to dispense the received liquid sample into the collection chamber 212. The liquid collector 210 is couplable and decouplable from the collection chamber 212 (in this example, by a threaded connection).

Prior to use, the liquid collector 210 holds a reagent in a reagent cavity 214 in a sealed condition. Actuation of the liquid collector 210 (in this example, coupling the liquid collector 210 to the collection chamber 212) unseals the reagent cavity 214 such that the reagent drains from the reagent cavity 214 into the collection chamber 212. More specifically, in this example, when the liquid collector 210 is threaded onto the collection chamber 212, the liquid collector will push a translatable body 218 in the liquid collector 210 from a first condition (shown in the bottom image in FIG. 28) in which the reagent cavity 214 is sealed to a second condition (shown in the bottom image in FIG. 29) in which the reagent cavity 214 is unsealed and a drain is opened to allow the reagent to drain from the reagent cavity 214 into the collection chamber 212.

In this example, there are sliding seals between the translatable body 218 and interior portions of the liquid collector 210. More particularly, in this example, resilient ribs 220 on the translatable body 218 form a sliding seal against an inner wall of the liquid collector 210, and a resilient opening 222 in the translatable body 218 forms another sliding seal with a fluid channel 224 in the liquid collector 210. When the translatable body 218 slides proximally inside the liquid collector 210, ramped ribs 226 on the fluid channel 224 will stretch the resilient opening 222 to open drains between each of the ribs 226, allowing the reagent to drain from the reagent cavity 214 into the collection chamber 212.

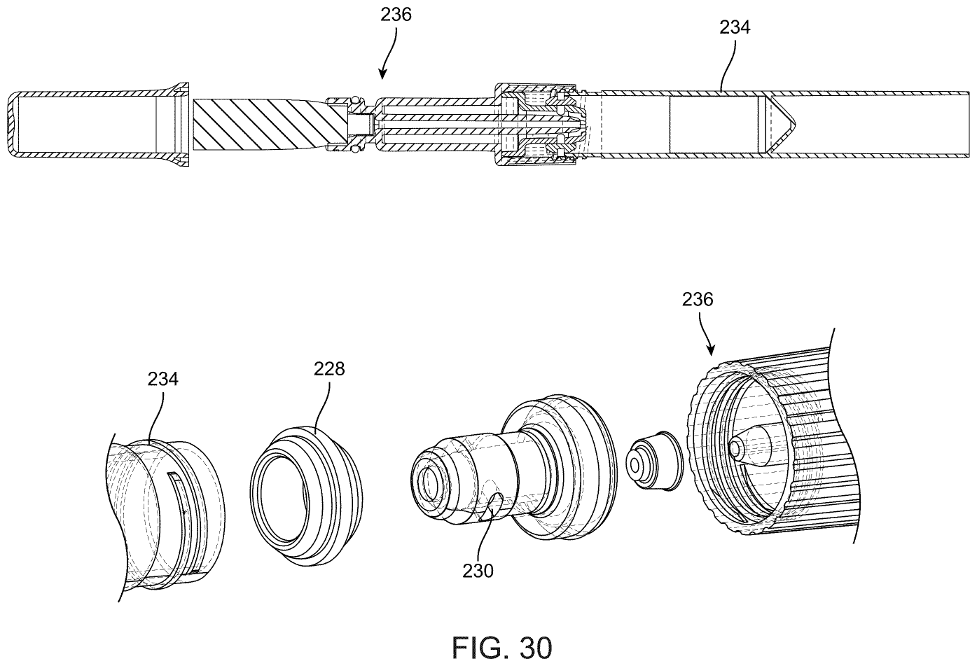

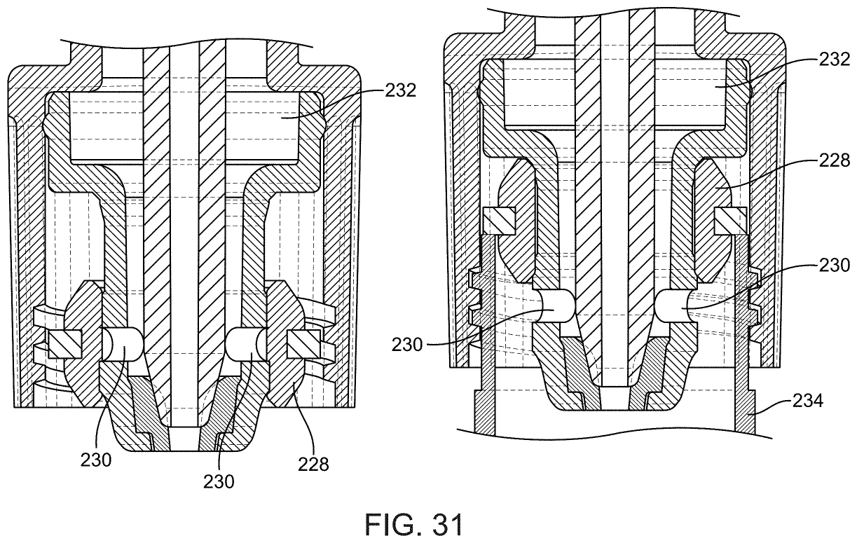

FIGS. 30-31 shows another example of a liquid sample collection system including a translatable body 228 that will open a drain 230 to unseal a reagent cavity 232 when the collection chamber 234 is coupled to the liquid collector 236. In this example, during coupling of the collection chamber 234 to the liquid collector 236, the collection chamber 234 will push the translatable body 228 in a proximal direction, uncovering drains 230 extending through a wall defining the reagent cavity 232, thereby allowing reagent to drain from the cavity 232 into the collection chamber 234.

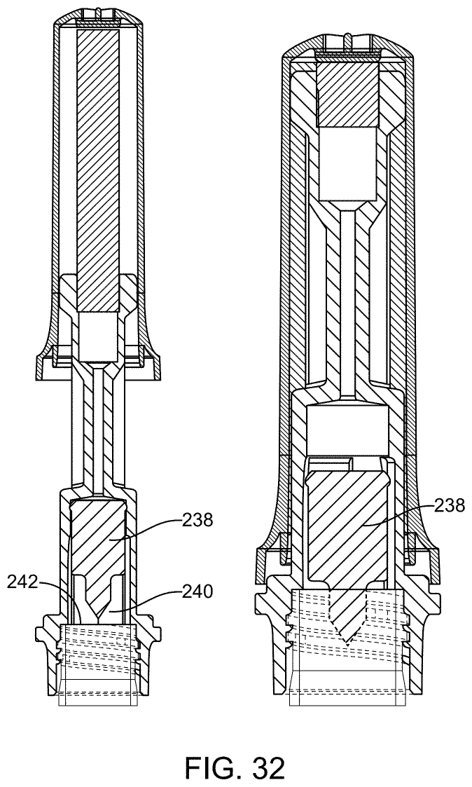

FIG. 32 shows another example of a liquid sample collection system that is similar to the system of FIG. 27 and includes a translatable plunger 238, except that in the system of FIG. 27 reagent is present in a reagent cavity 240 distal to the plunger 238 that is maintained in a sealed and preserved condition prior to use. As with the system of FIG. 27, actuating the liquid collector unseals the reagent by pushing the plunger 238 distally to disrupt a sealing membrane 242. In this example, when the plunger 238 disrupts the sealing membrane 242, reagent can then drain into the collection chamber.

The above description includes numerous examples of liquid collection devices, systems, and methods. These examples are not intended to be limiting. Additions, deletions, substitutions, and other modifications may be made to the examples described above without departing from the scope or spirit of the inventions set out in the following claims.

1. A liquid sample collection system comprising:
(a) a liquid collection device;
(b) a cap for the liquid collection device;
(c) a sealed reagent liquid container installed in the cap;
wherein depression of the cap relative to the liquid collective device unseals the reagent liquid container.

2. The liquid sample collection system of claim 1, wherein the liquid collection device comprises a collection pad and a collection chamber, wherein unsealing the reagent liquid container causes a reagent liquid to flow through the collection pad and into the collection chamber.

3. The liquid sample collection system of claim 2, wherein depression of the cap compresses the collection pad against the sealed reagent liquid container to unseal the reagent liquid container.

4. The liquid sample collection system of claim 3, wherein the collection pad collapses as it compresses against the sealed reagent liquid container.

5. The liquid sample collection system of claim 2, wherein the sealed reagent liquid container comprises a membrane, wherein compression of the collection pad against the sealed reagent liquid container dislodges the membrane.

6. The liquid sample collection system of claim 5, wherein the cap comprises a first open end for receiving the liquid collection device and a second open end for connecting to the sealed reagent liquid container.

7. The liquid sample collection system of claim 2, wherein compression of the collection pad against the sealed reagent liquid container translates the sealed reagent liquid container vertically inside the cap.

8. The liquid sample collection system of claim 7, wherein the sealed reagent liquid container is held inside the cap by a friction fit between the sealed reagent liquid container and the cap.

9. The liquid sample collection system of claim 8, wherein the cap further comprises an internal protrusion extending downward towards the sealed reagent liquid container, wherein compression of the collection pad against the sealed reagent liquid container translates the sealed reagent liquid container upward into contact with the internal protrusion to unseal the reagent liquid container.

10. The liquid sample collection system of claim 9, wherein the sealed reagent liquid container comprises a container body, a first membrane at one end of the container body facing the collection pad, and a second membrane at another end of the container body facing the internal protrusion, wherein depression of the cap onto the liquid collection device dislodges the first and second membranes.

11. The liquid sample collection system of claim 9, wherein the sealed reagent liquid container comprises an ampule.

12. A liquid sample collection system, comprising:
(a) a liquid collection device comprising: (i) a liquid collector, and (ii) a collection chamber, the liquid collector configured to receive a liquid sample and to dispense the received liquid sample into the collection chamber;
(b) wherein the liquid collector is couplable and de-couplable from the collection chamber; and
(c) wherein a sealing membrane seals the collection chamber;
(d) wherein the collection chamber holds a reagent; and
(e) wherein the liquid collector is actuatable to disrupt the sealing membrane.

13. The liquid sample collection system of claim 12, wherein actuation of the liquid collector to disrupt the sealing membrane comprises engaging or further engaging the liquid collector to the collection chamber.

14. The liquid sample collection system of claim 13, wherein the liquid collector comprises a liquid sample channel, and wherein the liquid sample channel is configured to pierce the sealing membrane when the liquid collector is engaged to or further engaged to the collection chamber.

15. The liquid sample collection system of claim 13, wherein the liquid collector comprises a cutting member configured to penetrate the sealing member along an arcuate path as the liquid collector is engaged to or further engaged to the collection chamber by rotating the liquid collector relative to the collection chamber.

16. The liquid sample collection system of claim 15, wherein the cutting member cuts a hanging disk from the sealing member as the liquid collector rotates relative to the collection chamber.

17. The liquid sample collection system of claim 13, wherein the sealing membrane is sealed across an opening of the collection chamber.

18. The liquid sample collection system of claim 13, further comprising an adapter, wherein the adapter is mounted to an opening of the collection chamber, wherein the sealing member is sealed across an opening of the adapter.

19. The liquid sample collection system of claim 18, wherein the liquid collector comprises a penetrating element that penetrates the sealing member and engages the liquid collector to the adapter such that removal of the liquid collector from the collection chamber also pulls the adapter out of the collection chamber.

20. The liquid sample collection system of claim 19, wherein the penetrating element comprises an expandable distal end that expands to engage the adapter after penetrating the sealing member.

21. The liquid sample collection system of claim 18, wherein the liquid collector comprises a lock that locks to the adapter as the liquid collector is engaged to or further engaged to the collection chamber such that disengagement of the liquid collector from the collection chamber also disengages the adapter from the liquid collection chamber.

22. The liquid sample collection system of claim 21, wherein the lock comprises part of a ratchet mechanism.

23. The liquid sample collection system of claim 12, wherein actuation of the liquid collector comprises depressing a portion of the liquid collector relative to the collection chamber.

24. The liquid sample collection system of claim 23, wherein the portion of the liquid collector comprises a plunger that is translatable relative to a body of the liquid collector.

25. The liquid sample collection system of claim 24, wherein the plunger further comprises a fluid path for the collected sample extending from a proximal end of the plunger to a distal end of the plunger.

26. The liquid sample collection system of claim 25, wherein the proximal end of the plunger is in fluid communication with a collection pad configured to collect the collected sample.

27. The liquid sample collection system of claim 25, wherein the plunger and the body further comprise a seal extending between the plunger and an interior wall of the body.

28. The liquid sample collection system of claim 25, wherein the liquid collector further comprises a cap translatable relative to the body, wherein the plunger and the cap further comprise a seal extending between the plunger and an interior wall of the cap.

29. The liquid sample collection system of claim 24, wherein the body comprises a liquid chamber proximal to the plunger, wherein the liquid sample collection system is configured such that collected sample in the liquid chamber of the body causes the plunger to translate distally to disrupt the membrane.

30. The liquid sample collection system of claim 29, wherein the body further comprises a flow channel along an exterior of the plunger that opens when the plunger translates distally relative to the body.

31. The liquid sample collection system of claim 29, wherein the liquid chamber of the body is fluidly connected to a reduced size fluid conduit extending proximally from the liquid chamber.

32. The liquid sample collection system of claim 12, wherein the liquid collector comprises a body including a first body portion and a second body portion, wherein actuation of the liquid collector comprises depressing the first body portion relative to the second body portion from a first condition in which the sealing membrane is not disrupted and a second condition in which the sealing membrane is disrupted.

33. The liquid sample collection system of claim 32, wherein the first and second body portions are rotationally fixed relative to one another at least when the first and second body portions are in the second condition.

34. The liquid sample collection system of claim 32, wherein the first and second body portions are rotationally fixed relative to one another when the first and second body portions are in the first and second conditions.

35. A liquid sample collection system, comprising:
(a) a liquid collection device comprising: (i) a liquid collector, and (ii) a collection chamber, the liquid collector configured to receive a liquid sample and to dispense the received liquid sample into the collection chamber;
(b) wherein the liquid collector is couplable and de-couplable from the collection chamber; and
(c) wherein the liquid collector holds a reagent in a reagent cavity of the liquid collector in a sealed condition; and
(d) wherein the liquid collector is actuatable to unseal the reagent such that the reagent drains from the reagent cavity to the collection chamber.

36. The liquid sample collection system of claim 35, wherein actuation of the liquid collector comprises coupling or further coupling the liquid collector to the collection chamber.

37. The liquid sample collection system of claim 36, wherein the liquid collector comprises a translatable body, wherein coupling or further coupling the liquid collector to the collection chamber translates the translatable body to open a drain and unseal the reagent cavity.

38. The liquid sample collection system of claim 37, wherein coupling or further coupling the liquid collector to the collection chamber causes a proximal end of the collection chamber to push the translatable body in a proximal direction.

39. The liquid sample collection system of claim 37, wherein the reagent cavity is defined by the translatable body and an additional interior surface of the liquid collector, wherein the liquid collector comprises a first sliding seal between the translatable body and the additional interior surface, wherein the liquid collector comprises a second sliding seal between the translatable body and an interior fluid channel of the liquid collector.

40. The liquid sample collection system of claim 39, wherein the interior fluid channel further comprises a ramped surface; wherein coupling or further coupling the liquid collector to the collection chamber causes the ramped surface to unseal the second sliding seal such that the reagent drains from the reagent cavity to the collection chamber.

41. The liquid sample collection system of claim 37, wherein coupling or further coupling the liquid collector to the collection chamber translates the translatable body to uncover the drain and unseal the reagent cavity.

42. The liquid sample collection system of claim 41, wherein coupling or further coupling the liquid collector to the collection chamber pushes the translatable body past the drain.

43. The liquid sample collection system of claim 35, wherein actuating the liquid collector to unseal the reagent such that the reagent drains from the reagent cavity comprises translating a first portion of the liquid collector relative to a second portion of the liquid collector.

44. The liquid sample collection system of claim 43, wherein the liquid collector further comprises a plunger and a membrane, the membrane sealing the reagent cavity, wherein actuating the liquid collector causes the plunger to translate and disrupt the membrane such that the reagent can drain into the collection chamber.

45. The liquid sample collection system of claim 44, wherein disrupting the membrane also opens a flow path for the liquid sample to flow into the collection chamber.

46. The liquid sample collection system of claim 43, wherein translating a first portion of the liquid collector relative to a second portion of the liquid collector comprises causing the liquid sample to flow into contact with the plunger such that the plunger translates distally to disrupt the membrane.

47. The liquid sample collection system of claim 46, wherein translating the plunger distally opens a flow channel along an exterior of the plunger such that the liquid sample can flow past the plunger and into the collection chamber.

48. The liquid sample collection system of claim 44, wherein actuating the liquid collector also opens a fluid path through the plunger for the collected liquid sample to flow into the collection chamber.
